(19) United States
(12) Patent Application Publication
(10) Pub. No.: US 2025/0204873 A1
(43) Pub. Date: Jun. 26, 2025

(54) X-RAY DIAGNOSTIC APPARATUS AND CONTROL METHOD OF X-RAY DIAGNOSTIC APPARATUS

(71) Applicant: CANON MEDICAL SYSTEMS CORPORATION, Otawara-shi (JP)

(72) Inventor: Daisuke SATO, Utsunomiya (JP)

(73) Assignee: CANON MEDICAL SYSTEMS CORPORATION, Otawara-shi (JP)

(21) Appl. No.: 18/985,481

(22) Filed: Dec. 18, 2024

(30) Foreign Application Priority Data
Dec. 25, 2023 (JP) 2023-217973
Dec. 9, 2024 (JP) 2024-214378

(51) Int. Cl. A61B 6/08; A61B 6/00; A61B 6/06; A61B 6/46; A61B 90/00

(52) U.S. Cl. CPC A61B 6/08; A61B 6/06; A61B 6/467; A61B 6/5294; A61B 6/542; A61B 2090/366

(57) ABSTRACT

According to one embodiment, An X-ray diagnostic apparatus includes an X-ray tube, an X-ray detector panel, processing circuitry, and a lighting field projector. The X-ray detector panel detects X-rays irradiated from the X-ray tube and transmitted through an object. The processing circuitry is configured to select one or more lighting fields from a plurality of lighting fields for AEC (Automatic Exposure Control) provided in the X-ray detector panel, or to set one or more lighting fields for AEC in the X-ray detector panel. The lighting field projector visibly projects information indicating the selected or set one or more areas corresponding to the one or more lighting fields on the object using visible light.

### CROSS-REFERENCE TO RELATED APPLICATIONS

This application claims the benefit of priority of Japanese Patent Application No. 2023-217973, filed Dec. 25, 2023, and Japanese Patent Application No. 2024-214378, filed Dec. 9, 2024, the entire contents of all of which are incorporated herein by reference.

### FIELD

Embodiments described herein relate generally to an X-ray diagnostic apparatus and a control method of an X-ray diagnostic apparatus.

### BACKGROUND

There are various types of X-ray diagnostic apparatuses, such as general X-ray imaging apparatus and X-ray TV apparatus. Among these apparatuses, a general X-ray imaging apparatus has a relatively simple configuration to perform X-ray imaging of the chest and the like. An X-ray TV apparatus can acquire X-ray radiographic images as still images, and can acquire X-ray fluoroscopic images as moving images so that imaging treatment using medical devices such as catheters, i.e., IVR (InterVentional Radiology), can be performed.

Such X-ray diagnostic apparatuses usually have an AEC (Automatic Exposure Control) function. The AEC function can generate properly exposed X-ray fluoroscopic images and X-ray radiographic images (hereinafter, both of which are collectively referred to as X-ray images), and can reduce the radiation exposure to the object.

To realize the AEC function, a lighting field, which is an area for detecting the X-ray dose, is provided in the radiation field, which is an area where X-rays are irradiated and X-ray images are generated.

The lighting field is preferably set within the region of the object to be imaged, in other words, within the region of interest (ROI) of the object. The lighting fields are conventionally provided at one or more predetermined locations on the X-ray detector panel (e.g., FPD: Flat Panel Detector), and the X-ray detector panel surface is marked with a mark or the like indicating the position and size of the lighting fields.

There are also X-ray diagnostic apparatuses that are configured to allow the user, such as a radiographer, to select the desired lighting field from the multiple lighting fields that have been provided in advance, taking into consideration the positional relationship with the ROI.

However, since the X-ray detector panel is hidden behind the object (e.g., patient) during object positioning and imaging, the user such as the radiographer cannot confirm the position of the lighting field relative to the ROI.

It is known to superimpose the selected lighting field on the camera image of the object captured with a visible light camera. However, this technique requires a visible light camera and a monitor device for displaying the camera image, and the patient may even refuse to be photographed with the visible light camera depending on the type of procedure or examination region.

Further, in cases where the user such as the radiographer is required to assist the patient during the examination, the user must check the lighting field displayed on the monitor device while assisting the patient, which is inconvenient.

### BRIEF DESCRIPTION OF THE DRAWINGS

FIG. 1 is an external view showing an example of a configuration of an X-ray diagnostic apparatus according to the first embodiment.

FIG. 2A is a block diagram showing an example configuration of the X-ray diagnostic apparatus according to the first embodiment.

FIG. 2B is a schematic diagram showing a positional relationship between a lighting field projection light and a radiation field projection light when an object is viewed from an X-ray tube.

FIG. 3A is an explanatory diagram showing an example in which a lighting field projection apparatus is provided outside an X-ray irradiation housing.

FIG. 3B is an explanatory diagram showing an example in which the lighting field projection apparatus is provided inside the X-ray irradiation housing.

FIG. 4 is a flowchart showing an example of a processing flow or workflow of the X-ray diagnostic apparatus according to the first embodiment.

FIG. 5 is a diagram showing the concept of operation of the X-ray diagnostic apparatus according to the first embodiment.

FIG. 6 is a flowchart showing an example of a processing flow or workflow of the X-ray diagnostic apparatus according to a variant of the first embodiment.

FIG. 7 is a diagram showing the concept of operation of the X-ray diagnostic apparatus according to the variant of the first embodiment.

FIG. 8A is a block diagram showing an example configuration of the X-ray diagnostic apparatus according to the second embodiment.

FIG. 8B is a schematic diagram showing the positional relationship between the lighting field projection light and the radiation field projection light when the object is viewed from the X-ray tube according to the second embodiment.

FIG. 9 is a flowchart showing an example of a processing flow or workflow of the X-ray diagnostic apparatus according to the second embodiment.

FIG. 10 is a diagram showing the concept of operation of the X-ray diagnostic apparatus according to the second embodiment.

FIG. 11A is a block diagram showing an example configuration of the X-ray diagnostic apparatus according to the third embodiment.

FIG. 11B is a schematic diagram showing the positional relationship between the lighting field projection light and the radiation field projection light when the object is viewed from the X-ray tube according to the third embodiment.

FIG. 12 is a flowchart showing an example of a processing flow or workflow of the X-ray diagnostic apparatus according to the third embodiment.

FIG. 13 is a diagram showing the concept of operation of the X-ray diagnostic apparatus according to the third embodiment.

FIG. 14 is a flowchart showing an example of a processing flow or workflow of the X-ray diagnostic apparatus according to the fourth embodiment.

FIG. 15 is a diagram showing the concept of operation of the X-ray diagnostic apparatus according to the fourth embodiment.

FIG. 16A is a block diagram showing an example configuration of the X-ray diagnostic apparatus according to the fifth embodiment.

FIG. 16B is a schematic diagram showing a positional relationship between the lighting field projection light and the radiation field projection light when the object is viewed from an X-ray tube according to the fifth embodiment.

FIG. 17 is a flowchart showing an example of a processing flow or workflow of the X-ray diagnostic apparatus according to the fifth embodiment.

FIG. 18 is a diagram showing the concept of operation of the X-ray diagnostic apparatus according to the fifth embodiment.

### DETAILED DESCRIPTION

Hereinbelow, a description will be given of an X-ray diagnostic apparatus and a control method of an X-ray diagnostic apparatus according to embodiments of the present invention with reference to the drawings.

According to one embodiment, An X-ray diagnostic apparatus includes an X-ray tube, an X-ray detector panel, processing circuitry, and a lighting field projector. The X-ray detector panel detects X-rays irradiated from the X-ray tube and transmitted through an object. The processing circuitry is configured to select one or more lighting fields from a plurality of lighting fields for AEC (Automatic Exposure Control) provided in the X-ray detector panel, or to set one or more lighting fields for AEC in the X-ray detector panel. The lighting field projector visibly projects information indicating the selected or set one or more areas corresponding to the one or more lighting fields on the object using visible light.

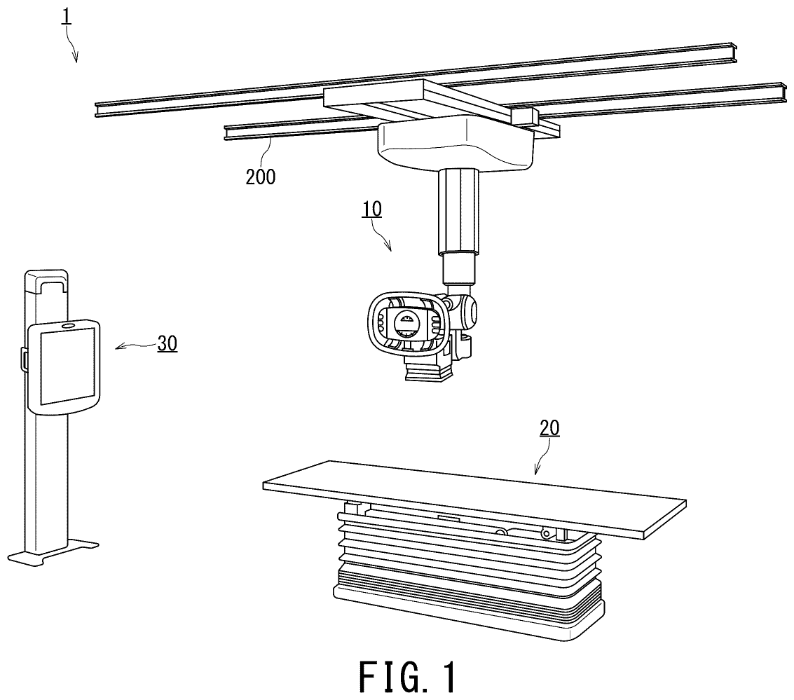

FIG. 1 is an external view showing an example of a configuration of an X-ray diagnostic apparatus 1 according to the first embodiment. The X-ray diagnostic apparatus 1 illustrated in FIG. 1 is an example of a general X-ray imaging apparatus that performs plain X-ray radiography of the chest and other parts of the body, but the X-ray diagnostic apparatus 1 is not limited to such apparatus and can include, for example, an X-ray TV system that can perform IVR. The X-ray diagnostic apparatus 1 includes, for example, an X-ray irradiation apparatus 10, a lying position imaging table 20, and a standing position imaging table 30.

The X-ray irradiation apparatus 10 is held by an X-ray tube holding apparatus. There are two types of X-ray tube holding apparatus, for example, a ceiling-type X-ray tube holding apparatus and a floor-mounted X-ray tube holding apparatus. FIG. 1 shows an exterior example of a ceiling-type X-ray tube holding apparatus. The ceiling-type X-ray tube holding apparatus has a ceiling rail 200, and the position of the holding X-ray irradiation apparatus 10 can be moved in parallel along the ceiling rail 200 in two orthogonal directions.

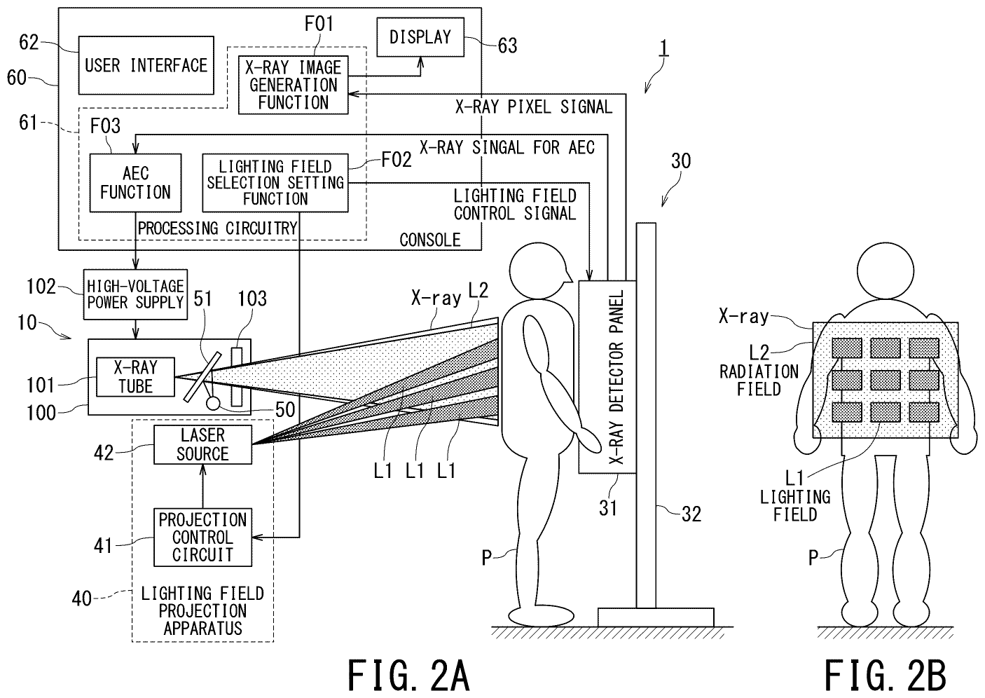

The standing position imaging table 30 is a device for imaging the object P (e.g., patient) in a standing position. The standing position imaging table 30 has an X-ray detector panel 31 (see FIG. 2). The X-rays irradiated from an X-ray tube 101 (see FIG. 2), which is housed in the X-ray irradiation apparatus 10, pass through the chest of object P and are detected by the X-ray detector panel 31.

Meanwhile, the lying position imaging table 20 is configured as a bed on which object P is placed in a lying position on a tabletop for imaging. X-rays irradiated from the X-ray tube 101 (see FIG. 2) housed in the X-ray irradiation apparatus 10 pass through the object P on the tabletop and are detected by the X-ray detector panel 31.

FIG. 2A is a block diagram showing an example configuration of the X-ray diagnostic apparatus 1 according to the first embodiment. The X-ray diagnostic apparatus 1 shown in FIG. 2A illustrates a configuration with the standing position imaging table 30, and the lying position imaging table 20 is omitted. In the following, the configuration and functions of the X-ray diagnostic apparatus 1 will be explained using an example in which object P is imaged by the standing position imaging table 30, but these explanations can also be applied to the case in which object P is imaged by the lying position imaging table 20.

As illustrated in FIG. 2A, the X-ray diagnostic apparatus 1 has the X-ray irradiation apparatus 10, the standing position imaging table 30, a console 60, and a lighting field projection apparatus 40.

The X-ray irradiation apparatus 10 has an X-ray tube 101 and a high-voltage power supply 102 that applies a high voltage to the X-ray tube 101. The X-ray tube 101 is housed in the X-ray irradiation housing 100. The X-ray irradiation housing 100 also houses an X-ray diaphragm apparatus 103. The X-ray diaphragm apparatus 103 has a plurality of movable diaphragm blades that form a movable aperture. The aperture formed by the diaphragm blades defines the irradiation range of X-rays irradiated from the X-ray tube 101 to the object P. The standing position imaging table 30 includes an X-ray detector panel 31 and a support stand 32 for supporting the X-ray detector panel 31.

The X-ray detector panel 31 has an X-ray detector such as an FPD. The X-ray detector panel 31 outputs X-ray pixel signals for generating X-ray images such as X-ray fluoroscopic images and X-ray radiographic images. The area of the X-ray detector panel 31 that detects X-rays passes through object P is referred to as the radiation field. In FIG. 2A, the area “X-ray” where X-rays passing through the object P irradiate the X-ray detector panel 31 is also illustrated. The area “X-ray” where X-rays irradiate the X-ray detector panel 31 and the radiation field, which is the area where X-rays passing through the object P are detected by the X-ray detector panel 31, are substantially the same.

The X-ray detector panel 31 also outputs an X-ray signal for AEC to realize the AEC function. The X-ray detector panel 31 has multiple areas called lighting fields, and X-ray signals detected in these lighting fields are output in real time as the X-ray signal for AEC.

FIG. 2B is a schematic diagram showing a positional relationship between a lighting field projection light L1 and a radiation field projection light L2 when an object P is viewed from the X-ray tube 101. L1 corresponds to the position and shape of the lighting field in the X-ray detector panel 31, and the radiation field projection light L2 corresponds to the position and shape of the radiation field (area where X-rays are radiated) in the X-ray detector panel 31. A predetermined number of lighting fields are initially set at predetermined locations within the radiation field. FIG. 2B shows an example of nine lighting fields arranged in three rows and three columns as the initial state.

In each lighting field, detector elements for AEC are provided to detect X-rays for AEC. These detector elements for AEC can be embedded in the FPD and configured as an integral part of the FPD. These detector elements for AEC can also be configured as external devices that are separate from the FPD.

As described below, each of these lighting fields can be deselected by manual operation by the user or by the automatic setting function of the device, or, conversely, the lighting field in the unselected state can be reselected.

In addition, as described below, instead of adjusting the lighting fields by deselecting and selecting multiple lighting fields that have been set in advance, it is possible to set one or more lighting fields of the desired shape in the X-ray detector panel 31.

The console 60 includes at least a user interface 62, a display 63, and processing circuitry 61.

The user interface 62 includes an input device that can be operated by a user and input circuitry that inputs signals from the input device. The input devices are realized, for example, by a control console, joystick, trackball mouse, keyboard, touch panel for input operation by touching the operation surface, touch screen that unifies the display screen and the touch pad, non-contact input circuitry using optical sensors, voice input circuitry, and the like.

The display 63 is a general display output device, such as a liquid crystal display or an OLED (Organic Light Emitting Diode) display, for example. The display 63 displays X-ray images such as X-ray fluoroscopic images and X-ray radiographic images generated by the processing circuitry 61, as well as various other data. All or part of the X-ray images and data displayed on the display 63 may be displayed on the touch panel or touch screen of the user interface 62.

The processing circuitry 61 has one or more processors and a memory. Each of the functions described below is realized through software processing by executing a program stored in the memory. In addition, the processing circuitry 61 can be configured to realize each function by hardware processing performed by an FPGA (Field Programmable Gate Array), ASIC (Application Specific Integrated Circuit), or the like. It can also be configured to realize each function by combining software processing and hardware processing.

As shown in FIG. 2A, the processing circuitry 61 realizes each function of an X-ray image generation function F01, a lighting field selection setting function F02, and an AEC function F03.

The X-ray image generation function F01 generates X-ray images such as X-ray fluoroscopic images and X-ray radiographic images based on the X-ray pixel signals output from the X-ray detector panel 31. The generated X-ray images may be displayed on the display 63.

The AEC function F03 performs AEC based on the X-ray signal for AEC output from the lighting field of the X-ray detector panel 31. For example, the average or maximum value of the X-ray signal for AEC output from multiple lighting fields is accumulated. When the accumulated values exceed a predetermined threshold value, The AEC function F03 instructs the high-voltage power supply 102 to stop the application of high voltage, thereby stopping X-ray irradiation. Such AEC function allows X-ray images to be generated as properly exposed images and also reduces the radiation exposure to the object.

The lighting field selection setting function F02 selects one or more lighting fields from the plurality of lighting fields for AEC provided in the X-ray detector panel 31, or sets one or more lighting fields for AEC in the X-ray detector panel 31. The information about the lighting field selected or set by the lighting field selection setting function F02 is output to the X-ray detector panel 31 as a lighting field control signal. The X-ray detector panel 31 adjusts the lighting field by deselecting and reselecting unselected lighting fields according to the lighting field control signal. More specific operations of the lighting field selection setting function F02 are described below.

The lighting field control signal generated by the lighting field selection setting function F02 is also sent to the lighting field projection apparatus 40. The lighting field projection apparatus 40 projects information indicating one or more areas corresponding to the lighting field selected or set in the lighting field selection setting function F02 onto the object P using visible light, e.g., laser light. In this embodiment, an example of using the lighting field projection light L1 as information indicating the one or more areas corresponding to the one or more selected or set lighting fields will be described.

The lighting field projection apparatus 40 has, for example, laser sources 42 and a projection control circuit 41. the laser sources 42 is positioned adjacent to the x-ray irradiation housing 100 that houses the x-ray tube 101. The projection control circuit 41 controls the on/off of each laser source 42 such that the laser lights emitted from the laser sources 42 are projected onto the object P as a lighting field projection lights L1 corresponding to the selected or set lighting field. The light sources for generating the lighting field projection light L1 is not limited to the laser sources 42, but can be any visible light source. When each of the laser source 42 has a shutter, the projection control circuit 41 may project the lighting field projection light L1 corresponding to the selected or set lighting field area onto the object P by switching the open/close state of each shutter 42b.

In addition to the lighting field projection light L1, a radiation field projection light L2 indicating the area of X-ray irradiation to the object P is also projected onto the object P. The radiation field projection light L2 is generated by the radiation field lamp 50 housed in the X-ray irradiation housing 100. The visible light generated by the radiation field lamp 50 is emitted toward the object P via the half mirror 51 and the aperture of the X-ray diaphragm apparatus 103, whereby the radiation field projection light L2 corresponding to the X-ray irradiation area is projected onto the object P. The radiation field lamp 50 and the half mirror 51 constitute the radiation field projector.

FIGS. 2A and 2B illustrate a situation in which the front surface of the chest of the standing object P is in contact with the X-ray detector panel 31, and X-rays are irradiated from the back side of the object P. In such a positioning of the object P, the user such as a radiographer cannot directly see the lighting fields on the surface of the X-ray detector panel 31, the user cannot see the lighting fields directly even when a lighting field is displayed on the surface of the X-ray detector panel 31 because the X-ray detector panel 31 is hidden behind the object P. In contrast, in the X-ray diagnostic apparatus 1 according to the present embodiment, the radiation field projection lights L2 and the lighting field projection light L1 are superimposed and projected onto the object P. As a result, the user viewing the object P from the X-ray irradiation apparatus 10 side can easily grasp the positional relationship between the lighting field and the radiation field that is currently selected or set, even though the position and number of the lighting fields can be adjusted in the X-ray diagnostic apparatus 1.

The method of projecting the lighting field and the radiation field onto the object P in a mutually distinguishable manner such that the positional relationship between the lighting fields and the radiation field is easily visible may be, for example, to change the color of the light between the radiation field projection light L2 and the lighting field projection lights L1, to change the intensity of the lights, to highlight the outer frame of one lighting area or to highlight the outer frames of both areas in a different manner, or a combination of these methods.

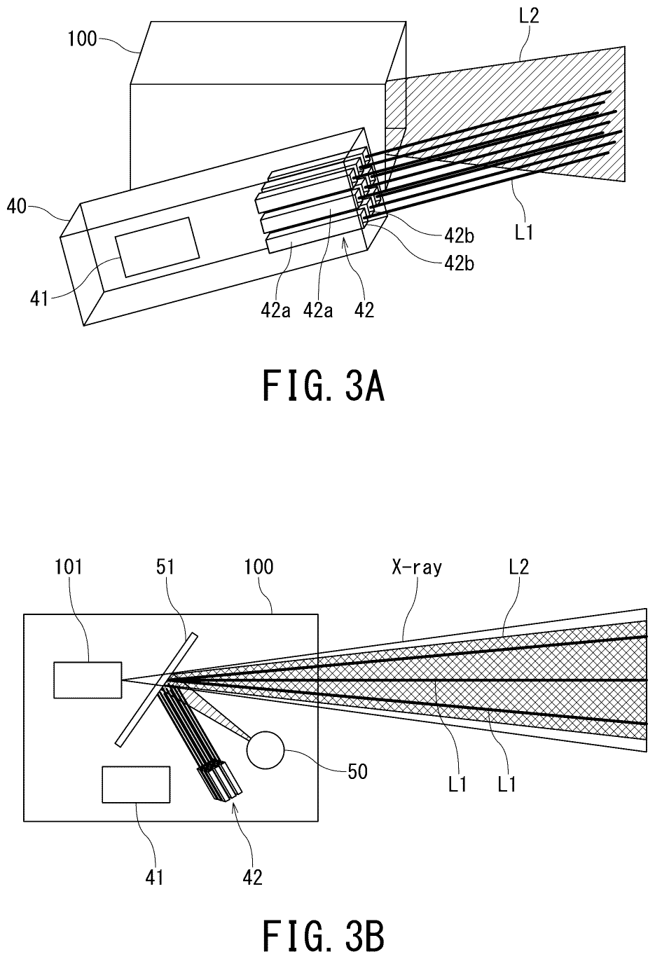

FIG. 3A is an explanatory diagram showing an example in which the lighting field projection apparatus 40 is provided outside the X-ray irradiation housing 100, and FIG. 3B is an explanatory diagram showing an example in which the lighting field projection apparatus is provided inside the X-ray irradiation housing 100.

The projection control circuit 41 switches the lighting state of each of the laser sources 42 such that the lighting field projection lights L1 corresponding to the selected or set lighting fields are projected onto the object P (see FIGS. 3A and 3B).

The enclosure 42a of each of the laser sources 42 may be provided with a shutter 42b. In this case, the projection control circuit 41 switches the open/close state of each of the shutters 42b such that information indicating areas, i.e., the lighting field projection lights L1, corresponding to the selected or set lighting fields are projected onto the object P, while information indicating other lighting field is not projected onto the object P.

When the laser sources 42 are provided inside the X-ray irradiation housing 100, as shown in FIG. 3B, the laser lights emitted from the laser sources 42 may be emitted toward the object P via the half mirror 51 and the aperture of the X-ray diaphragm apparatus 103, similar to the visible light generated by the radiation field lamp 50. In this case, the projection control circuit 41 may also switch the respective lighting states of each of the laser sources 42 or the open/close states of each of the shutters 42b such that the lighting field projection lights L1 corresponding to the selected or set lighting fields are projected onto the object P. The respective open/close states of the 42 laser sources or the 42b shutters may be switched.

The number of the laser sources 42 may be the same as the number of selectable or settable lighting fields (e.g., 6×6), or may be greater than the number of the lighting fields (e.g., 9×9). When there are more laser sources 42 than the number of the lighting fields that can be selected or set, for example, the correspondence between each of the lighting fields and each of the laser sources 42 can be set in advance. In this case, one laser source 42 may correspond to one lighting field, or one or more laser sources 42 may correspond to one lighting field.

The projection control circuit 41 may adjust the angle of incidence of the lighting field projection lights L1 incident on the object P by adjusting the angle of the laser sources 42. For example, when the distance between the x-ray irradiation housing 100 and the x-ray detector panel 31 is changed, the position of the laser beam of the laser source 42, which is incident obliquely to the object P, is shifted according to the changed distance. The projection control circuit 41 may adjust the angle of incidence of the lighting field projection lights L1 incident on the object P by adjusting the angle of the laser source 42 to correct this shift.

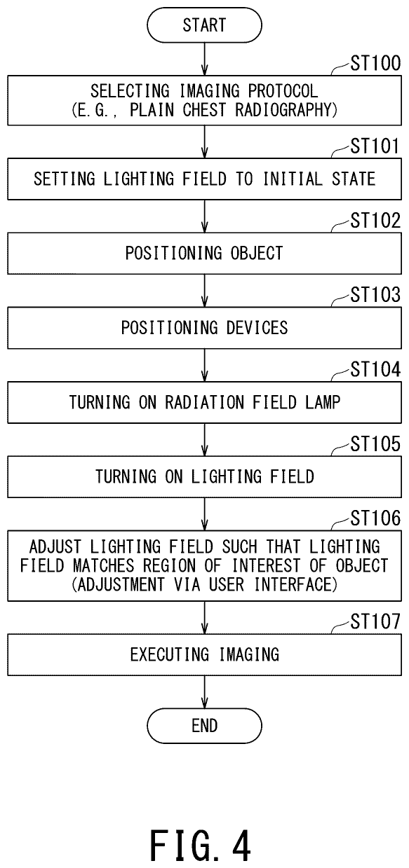

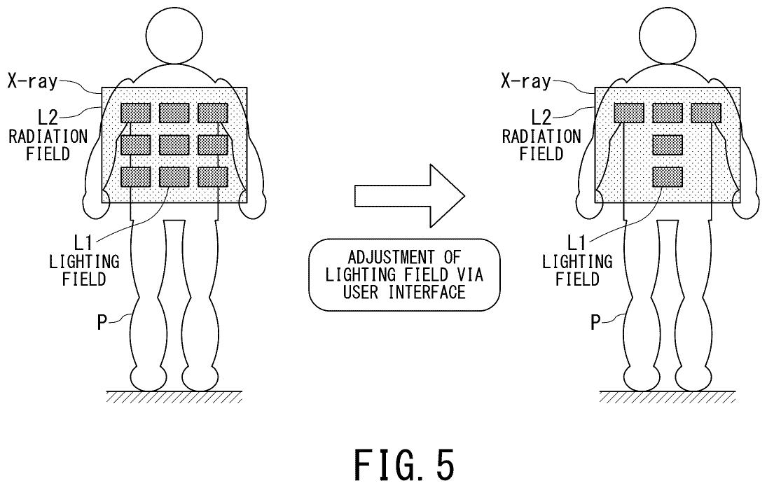

FIG. 4 is a flowchart showing an example of a processing flow or workflow of the X-ray diagnostic apparatus 1 according to the first embodiment. FIG. 5 is a diagram showing the concept of operation of the X-ray diagnostic apparatus 1 according to the first embodiment.

First, in step ST100, an imaging protocol, e.g., plain chest radiography, indicating a target region for imaging and the purpose of imaging, is set by the user via the user interface 62.

In step ST101, the lighting field is set to a preset initial state. For example, as shown in the left side of FIG. 5, all of the three rows and three columns of the lighting fields set in the X-ray detector panel 31 are selected as the initial state.

Next, positioning of the object is performed in step ST102, and positioning of the device is performed in step ST103. For example, the X-ray tube 101 is positioned at the back side of the object P.

Then, in step ST104, the radiation field lamp 50 is turned on. Accordingly, the radiation field projection light L2 is projected onto the object P.

In the next step ST105, the lighting field projection is turned on. Specifically, the laser sources 42 for the lighting fields is turned on and the lighting field projection lights L1 are projected onto the object P. The lighting field projection lights L1 projected at this stage corresponds to the initial state of the lighting fields, as shown in the left side of FIG. 5.

Next, in step ST106, the selection of the lighting field is adjusted such that the selected lighting fields match the region of interest of the object P. For example, while the user observes the lighting field projection lights L1 projected on the object P and the region of interest of the object P, the user can, via the user interface 62, deselect the lighting field that is set as the initial state, or reselect the unselected lighting field to adjust the lighting field.

The right side of FIG. 5B shows an example of the lighting fields after adjustment. For example, out of each lighting field in the initial state, the lighting field whose parts do not overlap with objects are decided to be inappropriate for AEC, and adjustments are made such that such lighting fields are deselected.

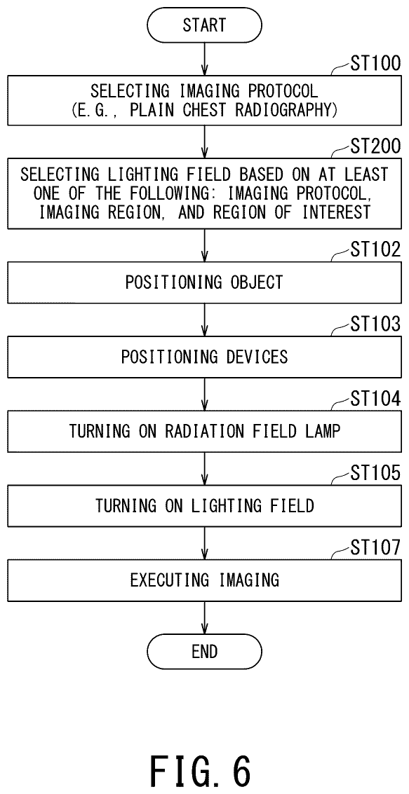

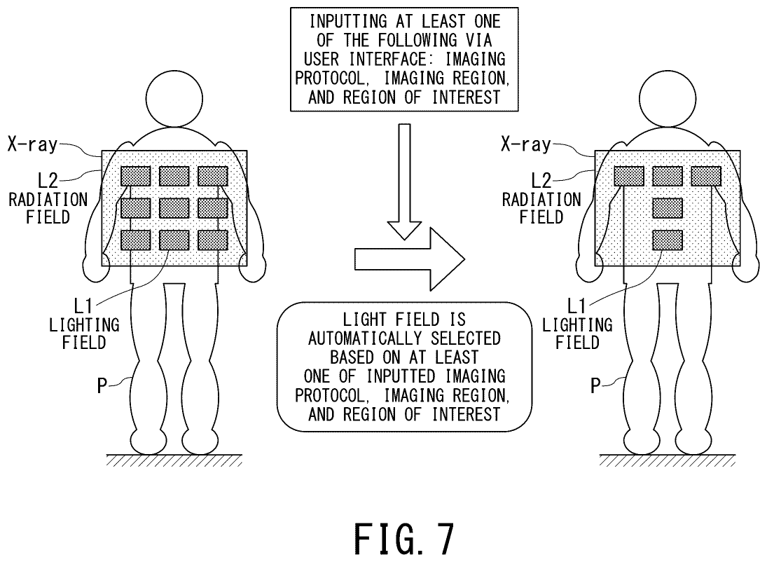

FIG. 6 is a flowchart showing an example of a processing flow or workflow of the X-ray diagnostic apparatus 1 according to a variant of the first embodiment. FIG. 7 is a diagram showing the concept of operation of the X-ray diagnostic apparatus 1 according to the variant of the first embodiment.

In FIG. 6, the same process as in the first embodiment (see flowchart in FIG. 4) is marked with the same numeral, and the explanation is omitted to avoid duplication. The same applies to the flowcharts of each of the following embodiments.

The difference between the processing of the first embodiment variant (see FIG. 6) and the processing of the first embodiment (see FIG. 4) is that the device automatically selects the lighting fields in step ST200 of FIG. 6, instead of manually adjusting the lighting fields in step ST106 of FIG. 4.

Specifically, in step ST200, the lighting field selection setting function F02 of the processing circuitry 61 automatically selects one or more lighting fields based on at least one of the imaging protocol, imaging region of the object, and region of interest of the object input via user interface 62. The left side of FIG. 7 shows the set lighting fields in their initial state, and the right side of FIG. 7 shows the lighting fields after selection (i.e., after adjustment) by the lighting field selection setting function F02.

In the variant of the first embodiment, the operational burden of manually selecting or manually deselecting the lighting fields is reduced.

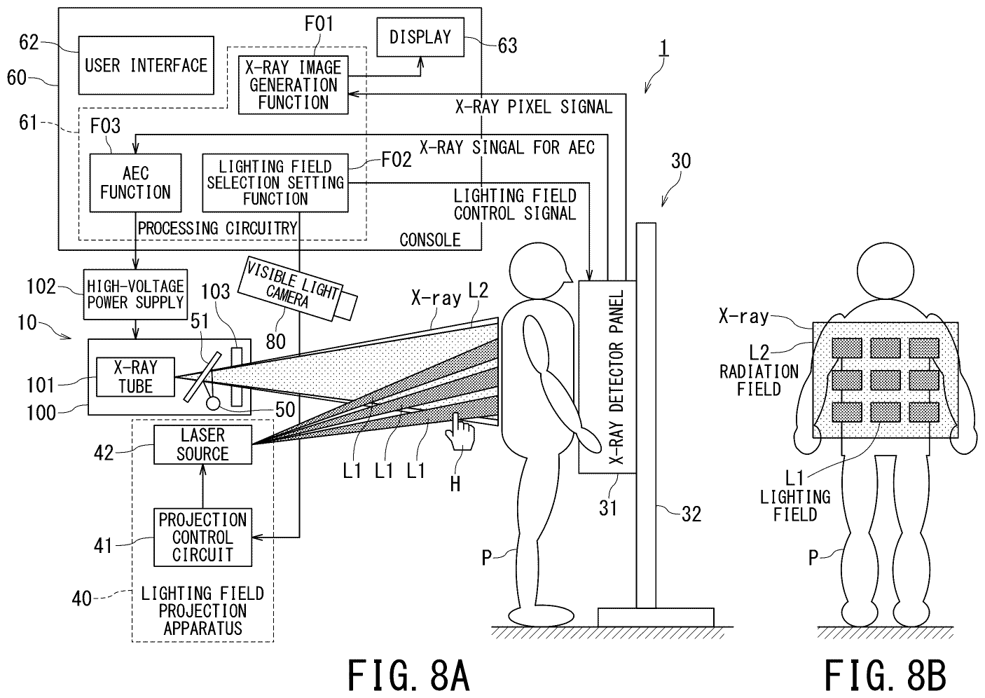

FIG. 8A is a block diagram showing an example configuration of the X-ray diagnostic apparatus 1 according to the second embodiment. FIG. 8B is a schematic diagram showing the positional relationship between the lighting field projection lights L1 and the radiation field projection light L2 when the object P is viewed from the X-ray tube 101 according to the second embodiment.

The difference between the second embodiment and the first embodiment is that the X-ray diagnostic apparatus 1 according to the second embodiment is provided with a visible light camera 80, as shown in FIG. 8A.

The visible light camera 80 captures a plurality of projected images on the object P, e.g., a plurality of lighting field projection lights L1, projected on the object P and a user gesture that specifies at least one of the plurality of lighting field projection lights L1. The user gesture is at least one of the position and movement of the user's arms and fingers, or the position and movement of a support member grasped by the user. In FIGS. 8A and 8B, the user's gesture is represented by the “hand” symbol H.

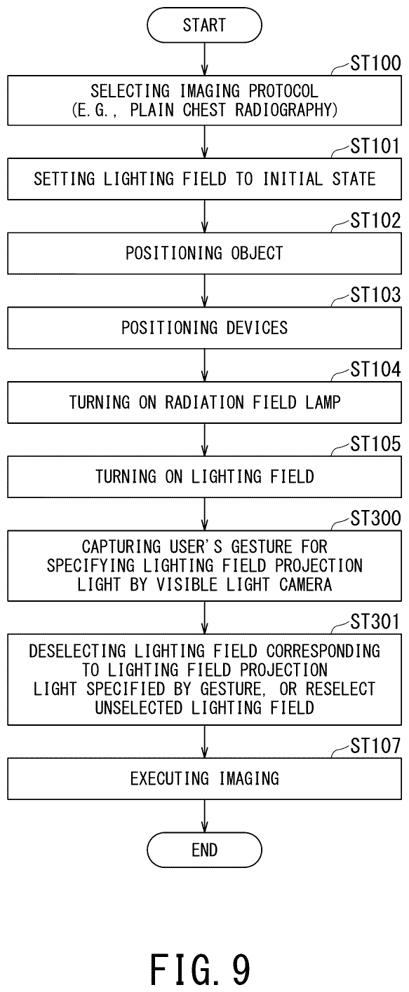

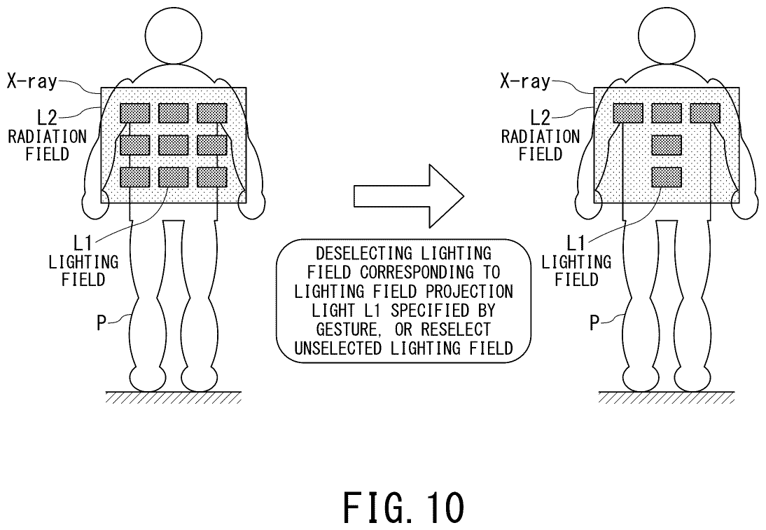

FIG. 9 is a flowchart showing an example of a processing flow or workflow of the X-ray diagnostic apparatus 1 according to the second embodiment. FIG. 10 is a diagram showing the concept of operation of the X-ray diagnostic apparatus 1 according to the second embodiment.

The difference between the processing of the second embodiment (see FIG. 9) and the first embodiment (see FIG. 4) is that instead of manual adjustment of the lighting field in step ST106 of FIG. 4, steps ST300 and ST301 are processed in the flowchart of FIG. 9.

In step ST300 of FIG. 9, the user's gesture to specify one of the lighting field projection lights L1 is captured by the visible light camera 80 and the captured image is sent to the processing circuitry 61.

In step ST301, the lighting field selection setting function F02 of the processing circuitry 61 identifies at least one lighting field projection light L1 pointed to by the user gesture based on the user gesture in the captured image of the visible light camera. Then the lighting field selection setting function F02 deselects the lighting field corresponding to at least one identified lighting field projection light L1 in the selected or set state in response to the gesture, or reselects the lighting field in the unselected state corresponding to at least one identified lighting field projection light L1.

The number and position of the lighting field projection lights L1 projected on the object P will also change synchronously with the adjustment of the lighting fields by the lighting field selection setting function F02.

According to the second embodiment of the X-ray diagnostic apparatus 1, manual operation via the user interface 62 is unnecessary, and the user can adjust the lighting fields while gazing at the object P.

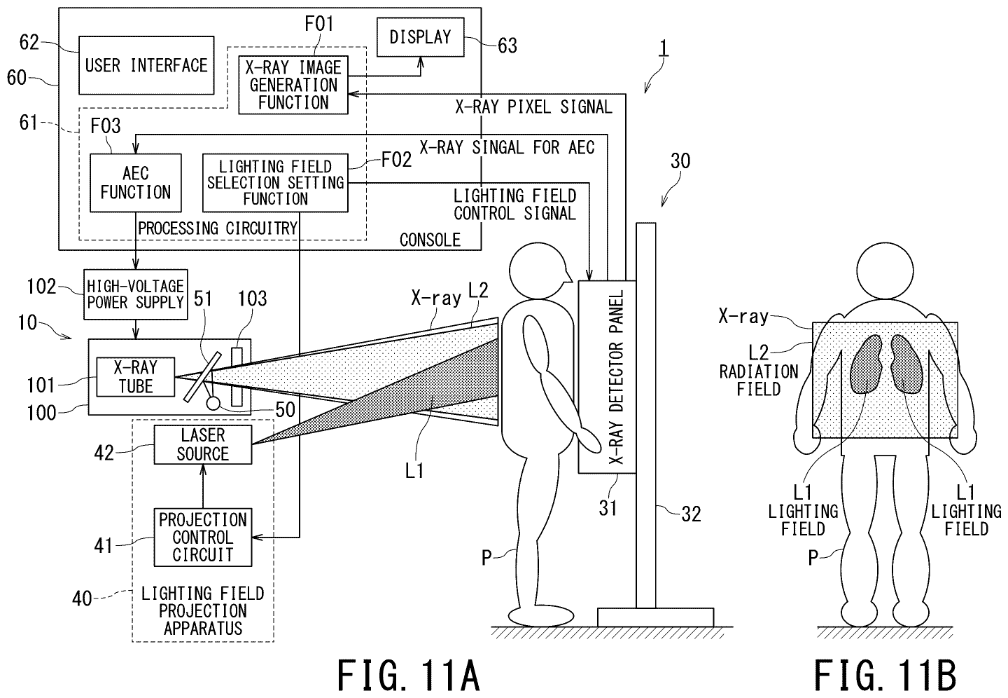

FIG. 11A is a block diagram showing an example configuration of the X-ray diagnostic apparatus 1 according to the third embodiment. FIG. 11B is a schematic diagram showing the positional relationship between the lighting field projection lights L1 and the radiation field projection light L2 when the object P is viewed from the X-ray tube 101 according to the third embodiment.

The configuration shown in the block diagrams of the third embodiment and the first embodiment is the same, but the method of arranging the lighting fields in the X-ray detector panel 31 and the method of setting the light fields by the lighting field selection setting function F02 of the processing circuitry 61 are different between the third embodiment and the first embodiment.

In the first embodiment (the same applies to the second embodiment), a plurality of lighting fields having a predetermined number, a predetermined position, and a predetermined shape are initially set in the X-ray detector panel 31, and for the lighting fields in the initial state, one or more lighting fields are deselected, or the deselected lighting fields are reselected to adjust the lighting fields.

In contrast, the third embodiment (as well as the fourth and fifth embodiments shown below) allows the setting of the lighting fields with the desired number, desired position, and desired predetermined shape within the X-ray detector panel 31, allowing for highly detailed and flexible adjustment of the lighting fields.

In FIGS. 11A and 11B, the lung field is assumed to be the region of interest of the object P, and the lighting fields corresponding to the shape of the lung field is set. FIGS. 11A and 11B show an example where the lighting field projection light L1 corresponding to the shape of the lung field is projected onto the object P by the laser source 42.

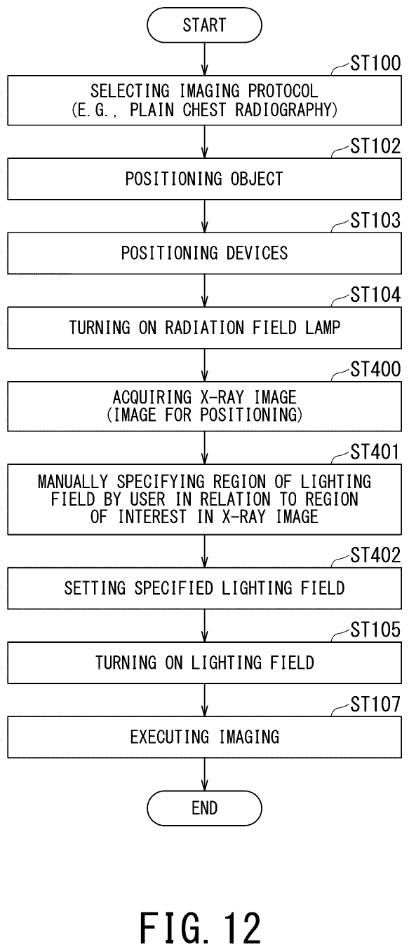

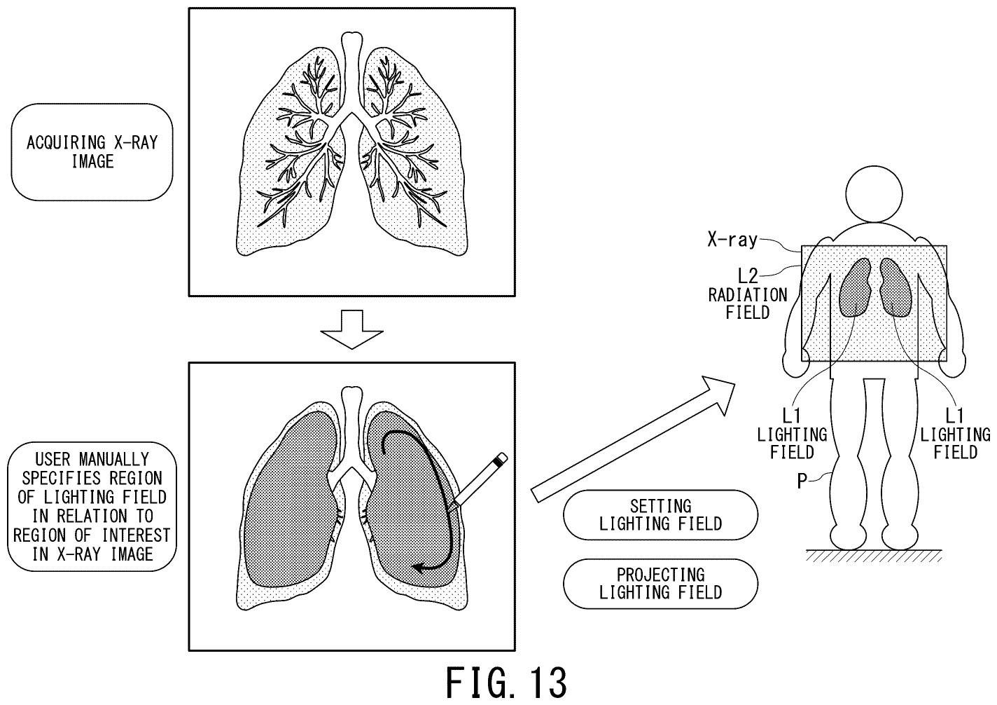

FIG. 12 is a flowchart showing an example of a processing flow or workflow of the X-ray diagnostic apparatus 1 according to the third embodiment. FIG. 13 is a diagram showing the concept of operation of the X-ray diagnostic apparatus 1 according to the third embodiment.

In step ST400, an X-ray image is acquired as an image for positioning the lighting fields. The X-ray image may be acquired, for example, as any frame image (still image) of relatively low dose X-ray fluoroscopic images. The acquired X-ray image may be displayed on the display 63 or a touch panel of the user interface 62, as illustrated in the upper left side of FIG. 13.

In step ST401, the user manually sets the region of the lighting field for the region of interest in the X-ray image. For example, as illustrated in the lower left side of FIG. 13, the X-ray image is displayed on the touch panel of the user interface 62, and the user manually sets the region of the lighting field by tracing around the lung field, which is the region of interest in the X-ray image, using a stylus pen, finger, or other indicating means. The information on the manually set region is acquired by the lighting field selection setting function F02 of the processing circuitry 61, and the lighting field selection setting function F02 specifies the position and shape of the lighting field based on the acquired information on the manually set region.

In step ST402, the lighting field selection setting function F02 sets the lighting field specified in this way on the X-ray detector panel 31. At the same time, the lighting field selection setting function F02 sets information about the specified lighting field to the projection control circuit 41 of the lighting field projection apparatus 40.

In step ST105, the lighting field projection is turned on based on the information about the set lighting field, and the lighting field projection light L1 is projected onto the object P as shown in the right side of FIG. 13.

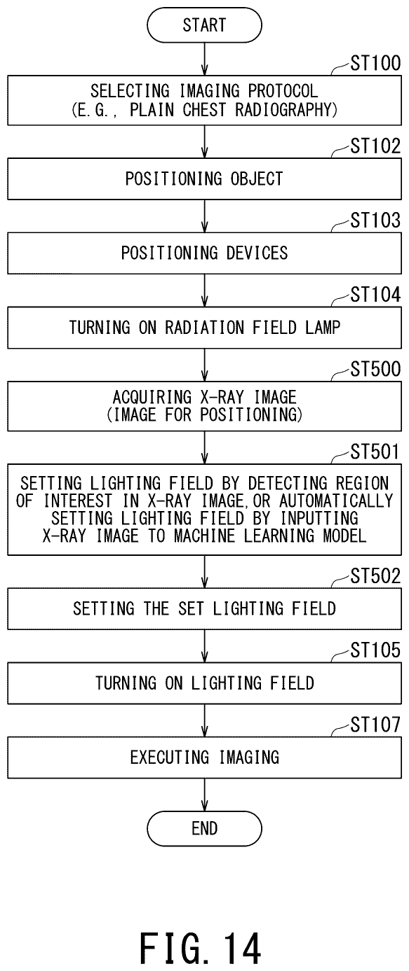

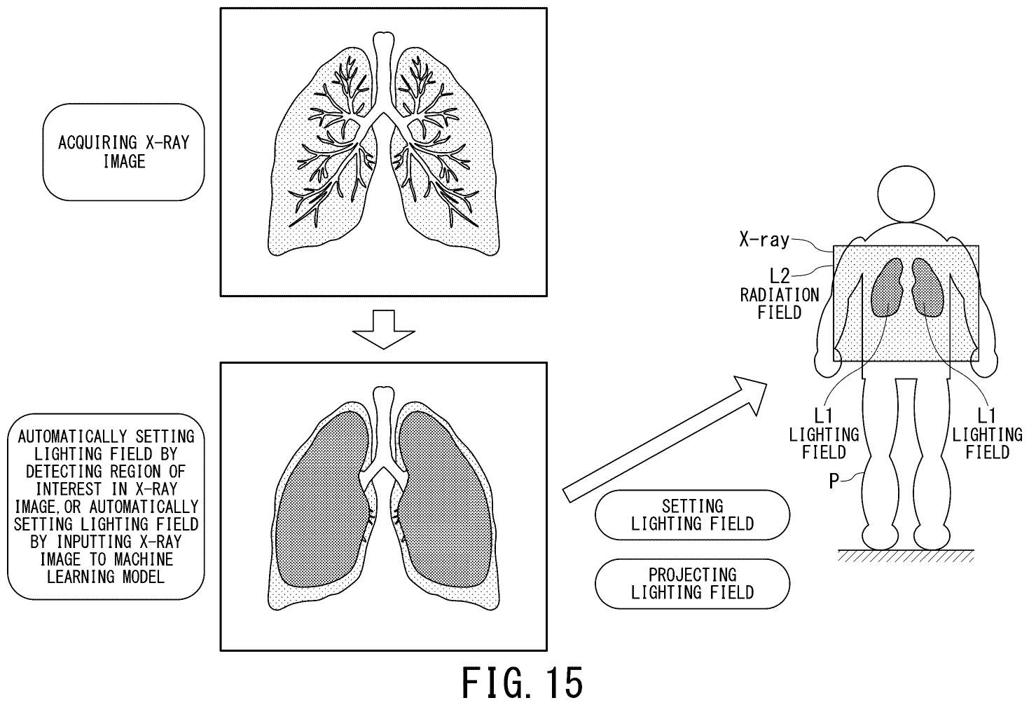

FIG. 14 is a flowchart showing an example of a processing flow or workflow of the X-ray diagnostic apparatus 1 according to the fourth embodiment. FIG. 15 is a diagram showing the concept of operation of the X-ray diagnostic apparatus 1 according to the fourth embodiment.

In step ST500 of FIG. 14, as in step ST400 of the third embodiment, an X-ray image is acquired as an image for positioning the lighting field, as shown in the upper left side of FIG. 15.

In the third embodiment, the lighting field is specified manually by the user for the region of interest in the X-ray image (see step ST402 in FIG. 12). In contrast, in the fourth embodiment, as shown in step ST501, the lighting field selection setting function F02 detects the region of interest of the object P based on the X-ray image, and automatically sets one or more lighting fields based on the detected region of interest (see lower left side of FIG. 15).

For example, the lighting field selection setting function F02 performs a known segmentation process on the X-ray image based on information about the imaging target included in the imaging protocol input in step ST100 (e.g., information that the imaging target is a lung field) to detect the region of interest of the object P. Then, based on the detected region of interest, the lighting field selection setting function F02 sets an area corresponding to the region of interest (e.g., an area substantially the same as the region of interest) as the lighting field.

Alternatively, the lighting field selection setting function F02 may automatically set one or more lighting fields by inputting the X-ray image acquired in step ST500 to a machine learning model generated in advance. For example, the such learned model generated in advance outputs information related to one or more lighting fields when an X-ray image is input.

In step ST502, as in the third embodiment, the lighting field selection setting function F02 sets the lighting field, that is automatically set using the learned model, in the X-ray detector panel 31, and sets information about the set lighting field in the projection control circuit 41 of the lighting field projection apparatus 40.

In step ST105, the lighting field projection is turned on based on the information about the set lighting field, and the lighting field projection light L1 is projected onto the object P, as shown in the right side of FIG. 15.

The X-ray diagnostic apparatus 1 according to the fourth embodiment, like the third embodiment, is capable of setting a detailed and highly flexible lighting field, and does not require the operational burden of specifying the region of the lighting field.

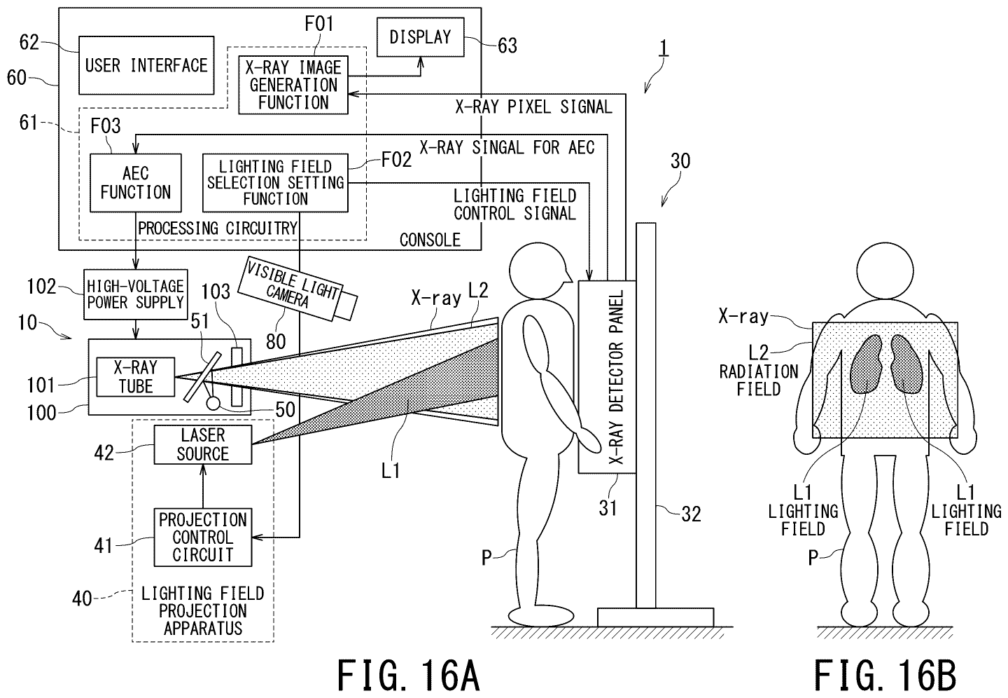

FIG. 16A is a block diagram showing an example configuration of the X-ray diagnostic apparatus 1 according to the fifth embodiment. FIG. 16B is a schematic diagram showing a positional relationship between the lighting field projection light L1 and the radiation field projection light L2 when the object P is viewed from the X-ray tube 101 according to the fifth embodiment.

The X-ray diagnostic apparatus 1 according to the fifth embodiment differs from the third and fourth embodiments in that it has the visible light camera 80 to capture the image of the object P.

In addition, in the fourth embodiment, the lighting field selection setting function F02 detects the region of interest of the object P based on the X-ray image of the object, and sets the lighting fields based on the detected region of interest. In contrast, in the fifth embodiment, the region of interest of the object P is detected based on the visible light image of the object P captured by the visible light camera 80, and sets the lighting field based on the detected region of interest.

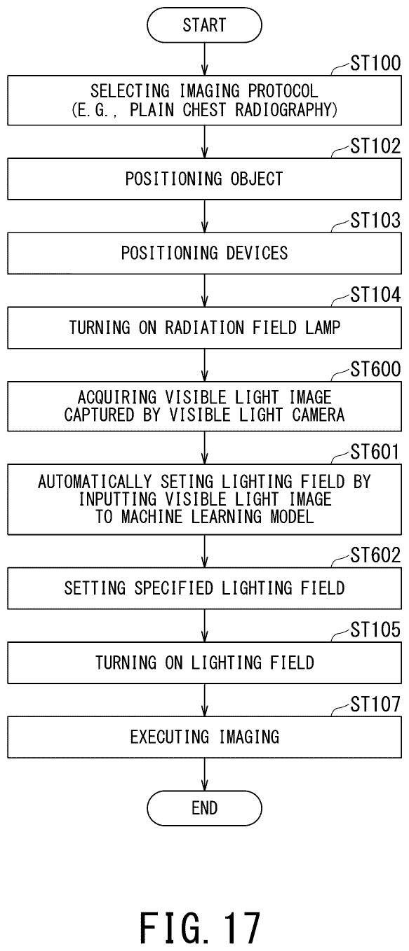

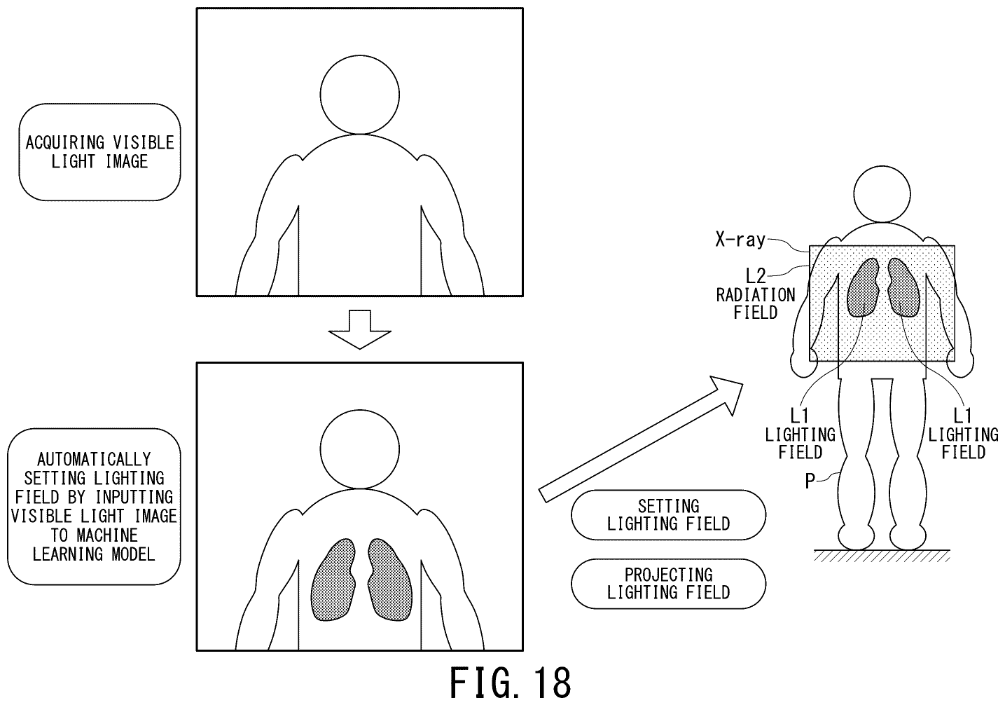

FIG. 17 is a flowchart showing an example of a processing flow or workflow of the X-ray diagnostic apparatus 1 according to the fifth embodiment. FIG. 18 is a diagram showing the concept of operation of the X-ray diagnostic apparatus 1 according to the fifth embodiment.

In step ST600 of FIG. 17, the visible light image is captured by the visible light camera 80, as shown in the upper left side of FIG. 18.

In the next step ST601, the lighting field selection setting function F02 automatically sets one or more lighting fields based on the captured visible light image. For example, the lighting field selection setting function F02 automatically sets one or more lighting fields by inputting the captured visible light image into a learned model generated in advance (see lower left side of FIG. 18).

Then, in step ST602, as in the third and fourth embodiments, the lighting field selection setting function F02 sets the lighting field, that is automatically set using the learned model, in the X-ray detector panel 31, and sets information about the set lighting field in the projection control circuit 41 of the lighting field projection apparatus 40.

In step ST105, the lighting field projection is turned on based on the information about the set lighting field, and the lighting field projection light L1 is projected onto the object P, as shown in the right side of FIG. 18.

Although the X-ray diagnostic apparatus 1 according to the fifth embodiment adds the configuration of the visible light camera 80 to the fourth embodiment, a visible light image captured by the visible light camera 80 is used instead of an X-ray image as the image used for automatic setting of the lighting field, thereby reducing radiation exposure to the object P.

According to at least one of the above-described embodiments, the user can easily visually recognize the lighting field for AEC in the X-ray diagnostic apparatus with a simple configuration.

While certain embodiments have been described, these embodiments have been presented by way of example only, and are not intended to limit the scope of the inventions. Indeed, the novel embodiments described herein may be embodied in a variety of other forms; furthermore, various omissions, substitutions, and changes in the form of the embodiments described herein may be made without departing from the spirit of the inventions. The accompanying claims and their equivalents are intended to cover such forms or modifications as would fall within the scope and spirit of the inventions.

With respect to the above embodiment, the following aspects are disclosed as one aspect and optional features of the invention.

### (Aspect 1)

An X-ray diagnostic apparatus, comprising:
an X-ray tube;
an X-ray detector panel detecting X-rays irradiated from the X-ray tube and transmitted through the object;
processing circuitry configured to select one or more lighting fields from a plurality of lighting fields for AEC (Automatic Exposure Control) provided in the X-ray detector panel, or to set one or more lighting fields for AEC in the X-ray detector panel; and
a lighting field projector visibly projecting one or more areas corresponding to the one or more lighting fields onto the object using visible light.

### (Aspect 2)

The X-ray diagnostic apparatus according to aspect 1, further comprising a user interface accepting input of an imaging protocol, an imaging region of the object, and a region of interest of the object,
wherein the processing circuitry is configured to select the one or more lighting fields from the plurality of lighting fields provided in advance, based on at least one of the inputted imaging protocol, the imaging region, and the region of interest.

### (Aspect 3)

The X-ray diagnostic apparatus according to aspect 1 or 2, wherein the processing circuitry is configured to select the one or more lighting fields from the plurality of lighting fields provided in advance, based on a specification by the user.

### (Aspect 4)

The X-ray diagnostic apparatus according to any one of aspects 1 to 3, further comprising a visible light camera capturing a plurality of projected images that corresponds to the plurality of lighting fields and is projected onto the object and, and capturing a gesture of the user to specify at least one of the projected images,
wherein the processing circuitry is configured to:
   identify the at least one projected image based on the gesture of the user from the captured image of the visible light camera; and
   deselect the lighting field in a selected state corresponding to the identified at least one projected image, or reselect the lighting field in an unselected state corresponding to the identified at least one projected image.

### (Aspect 5)

The X-ray diagnostic apparatus according to aspect 4, wherein the gesture of the user is at least one of the position and movement of the user's arm, finger or a support member grasped by the user.

### (Aspect 6)

The X-ray diagnostic apparatus according to any one of aspects 1 to 5, further comprising a radiation field projector projecting an area of a radiation field of the X-rays onto the object,
wherein the lighting field projector projects the information indicating the one or more areas corresponding to the one or more lighting fields onto the object such that the projected one or more areas are superimposed on the projected area of the radiation field and distinguishable from the projected area of the radiation field.

### (Aspect 7)

The X-ray diagnostic apparatus according to any one of aspects 1 to 6, further comprising:
an X-ray diaphragm that defines the radiation field of the X-rays, and
an X-ray irradiation housing that houses at least the X-ray tube and the X-ray diaphragm,
wherein a light source included in the lighting field projector for projecting an area of the lighting field is provided adjacent to the X-ray irradiation housing.

### (Aspect 8)

The X-ray diagnostic apparatus according to any one of aspects 1 to 7, further comprising:
a display displaying an X-ray image of the object acquired using the X-rays irradiated from the X-ray tube; and
a user interface accepting a specification of the one or more lighting fields for the region of interest in the X-ray image displayed on the display,
wherein the processing circuitry is configured to set one or more lighting fields for the AEC based on the one or more lighting fields specified by the user interface.

### (Aspect 9)

The X-ray diagnostic apparatus according to any one of aspects 1 to 8, wherein the processing circuitry is configured to detect the region of interest of the object based on the X-ray image of the object acquired using the X-rays irradiated from the X-ray tube, and set the one or more lighting fields for the AEC based on the detected region of interest.

### (Aspect 10)

The X-ray diagnostic apparatus according to any one of aspects 1 to 9, further comprising a user interface accepting input of an imaging protocol,
wherein the processing circuitry is configured to detect the region of interest of the object by a segmentation process based on information on an imaging target included in the input imaging protocol.

### (Aspect 11)

The X-ray diagnostic apparatus according to any one of aspects 1 to 10, wherein the processing circuitry is configured to set one or more lighting fields for the AEC by inputting an X-ray image of the object acquired using X-rays irradiated from the X-ray tube into a pre-generated trained model.

### (Aspect 12)

The X-ray diagnostic apparatus according to any one of aspects 1 to 11, further comprising a visible light camera to capture the object,
wherein the processing circuitry is configured to set one or more lighting fields for the AEC by inputting a visible light image of the object captured by the visible light camera into a pre-generated trained model.

### (Aspect 13)

A control method of an X-ray diagnostic apparatus, the X-ray diagnostic apparatus at least including an X-ray tube and an X-ray detector panel to detect X-rays irradiated from the X-ray tube and transmitted through the object, comprising:
selecting one or more lighting fields from a plurality of lighting fields for AEC (Automatic Exposure Control) provided in the X-ray detector panel, or to set one or more lighting fields for AEC in the X-ray detector panel; and
visibly projecting one or more areas corresponding to the one or more lighting fields onto the object using visible light.

What is claimed is:

1. An X-ray diagnostic apparatus, comprising:
an X-ray tube;
an X-ray detector panel detecting X-rays irradiated from the X-ray tube and transmitted through an object;
processing circuitry configured to select one or more lighting fields from a plurality of lighting fields for AEC (Automatic Exposure Control) provided in the X-ray detector panel, or to set one or more lighting fields for AEC in the X-ray detector panel; and
a lighting field projector visibly projecting information indicating one or more areas corresponding to the one or more lighting fields onto the object using visible light.

2. The X-ray diagnostic apparatus according to claim 1, further comprising a user interface accepting input of an imaging protocol, an imaging region of the object, and a region of interest of the object,
wherein the processing circuitry is configured to select the one or more lighting fields from the plurality of lighting fields provided in advance, based on at least one of the inputted imaging protocol, the imaging region, and the region of interest.

3. The X-ray diagnostic apparatus according to claim 1, wherein the processing circuitry is configured to select the one or more lighting fields from the plurality of lighting fields provided in advance, based on a specification by a user.

4. The X-ray diagnostic apparatus according to claim 3, further comprising a visible light camera capturing a plurality of projected images that corresponds to the plurality of lighting fields and is projected onto the object and, and capturing a gesture of the user to specify at least one of the projected images,
wherein the processing circuitry is configured to:
identify the at least one projected image based on the gesture of the user from the captured image of the visible light camera; and
deselect the lighting field in a selected state corresponding to the identified at least one projected image, or reselect the lighting field in an unselected state corresponding to the identified at least one projected image.

5. The X-ray diagnostic apparatus according to claim 4, wherein the gesture of the user is at least one of a position and movement of the user's arm, finger or a support member grasped by the user.

6. The X-ray diagnostic apparatus according to claim 1, further comprising a radiation field projector projecting an area of a radiation field of the X-rays onto the object,
wherein the lighting field projector projects the information indicating the one or more areas corresponding to the one or more lighting fields onto the object such that the projected one or more areas are superimposed on the projected area of the radiation field and distinguishable from the projected area of the radiation field.

7. The X-ray diagnostic apparatus according to claim 1, further comprising:
an X-ray diaphragm that defines a radiation field of the X-rays, and
an X-ray irradiation housing that houses at least the X-ray tube and the X-ray diaphragm,
wherein the lighting field projector includes a plurality of light sources to project the information indicating the one or more areas corresponding to the one or more lighting fields, the light sources being provided in a number corresponding at least to a number of the lighting fields, and is provided adjacent to an exterior of the X-ray irradiation housing or inside the X-ray irradiation housing.

8. The X-ray diagnostic apparatus according to claim 7, wherein the lighting field projector includes a projection control circuit that is configured to switch a lighting state of each of the plurality of the light sources such that information indicating one or more areas corresponding to the one or more lighting fields selected or set by the processing circuitry is projected onto the object.

9. The X-ray diagnostic apparatus according to claim 8, wherein the lighting field projector includes the plurality of the light sources, and the number of the light sources is greater than the number of the lighting fields.

10. The X-ray diagnostic apparatus according to claim 8, wherein:
the lighting field projector includes a shutter that shields a light path of the each of the plurality of the light sources; and
the projection control circuit of the lighting field projector is configured to switch an open/close state of the shutter of the each of the plurality of light sources such that the information indicating the one or more areas corresponding to the one or more lighting field areas selected or set by the processing circuitry is projected onto the object and information indicating an area corresponding to other lighting field is not projected onto the object.

11. The X-ray diagnostic apparatus according to claim 1, further comprising:
a display displaying an X-ray image of the object acquired using the X-rays irradiated from the X-ray tube; and
a user interface accepting a specification of the one or more lighting fields for a region of interest in the X-ray image displayed on the display,
wherein the processing circuitry is configured to set one or more lighting fields for the AEC based on the one or more lighting fields specified by the user interface.

12. The X-ray diagnostic apparatus according to claim 1, wherein the processing circuitry is configured to detect a region of interest of the object based on an X-ray image of the object acquired using the X-rays irradiated from the X-ray tube, and set the one or more lighting fields for the AEC based on the detected region of interest.

13. The X-ray diagnostic apparatus according to claim 12, further comprising a user interface accepting input of an imaging protocol,
wherein the processing circuitry is configured to detect the region of interest of the object by a segmentation process based on information on an imaging target included in the input imaging protocol.

14. The X-ray diagnostic apparatus according to claim 1, wherein the processing circuitry is configured to set one or more lighting fields for the AEC by inputting an X-ray image of the object acquired using X-rays irradiated from the X-ray tube into a pre-generated trained model.

15. The X-ray diagnostic apparatus according to claim 1, further comprising a visible light camera to capture the object,
wherein the processing circuitry is configured to set one or more lighting fields for the AEC by inputting a visible light image of the object captured by the visible light camera into a pre-generated trained model.

16. A control method of an X-ray diagnostic apparatus, the X-ray diagnostic apparatus at least including an X-ray tube and an X-ray detector panel to detect X-rays irradiated from the X-ray tube and transmitted through an object, comprising:
selecting one or more lighting fields from a plurality of lighting fields for AEC (Automatic Exposure Control) provided in the X-ray detector panel, or to set one or more lighting fields for AEC in the X-ray detector panel; and
visibly projecting information indicating one or more areas corresponding to the one or more lighting fields onto the object using visible light.

17. The control method of the X-ray diagnostic apparatus according to claim 16, wherein:
the X-ray diagnostic apparatus includes a user interface to accept input of an imaging protocol, an imaging region of the object, and a region of interest of the object; and
the setting step includes selecting step to select the one or more lighting fields from the plurality of lighting fields provided in advance, based on at least one of the inputted imaging protocol, the imaging region, and the region of interest.

18. The control method of the X-ray diagnostic apparatus according to claim 16, wherein the setting step includes selecting step to select the one or more lighting fields from the plurality of lighting fields provided in advance, based on a specification by a user.

19. The control method of the X-ray diagnostic apparatus according to claim 16, further comprising projecting an area of a radiation field of the X-rays onto the object,
wherein the visibly projecting step includes projecting step to project the information indicating the one or more areas corresponding to the one or more lighting fields onto the object such that the projected one or more areas are superimposed on the projected area of the radiation field and distinguishable from the projected area of the radiation field.

20. The control method of the X-ray diagnostic apparatus according to claim 16, wherein:
the X-ray diagnostic apparatus includes a display displaying an X-ray image of the object acquired using the X-rays irradiated from the X-ray tube; and a user interface accepting a specification of the one or more lighting fields for a region of interest in the X-ray image displayed on the display; and
the setting step includes setting step to set one or more lighting fields for the AEC based on the one or more lighting fields specified by the user interface.
